(19) United States
(12) Patent Application Publication
(10) Pub. No.: US 2012/0009153 A1
(43) Pub. Date: Jan. 12, 2012

(54) COMPOSITIONS FOR BACTERIAL MEDIATED GENE SILENCING AND METHODS OF USING THE SAME

(76) Inventors: Hongnian Guo, Brookline, MA (US); Chiang J. Li, West Roxbury, MA (US); Johannes Fruehauf, Boston, MA (US)

(21) Appl. No.: 12/856,437

(22) Filed: Aug. 13, 2010

Related U.S. Application Data
(60) Provisional application No. 61/233,630, filed on Aug. 13, 2009.

(51) Int. Cl. A61K 35/74; C12N 15/74; A61P 31/12; A61P 35/00; A61P 29/00; A61P 31/04; C12N 5/071; C12N 1/21

(52) U.S. Cl. 424/93.2; 435/366; 435/320.1; 435/252.3

(57) ABSTRACT

The invention features compositions and methods for delivering small interfering (siRNAs), e.g., shRNAs, to host cells using non-pathogenic strains of Salmonella bacteria containing nucleic acid expression constructs encoding shRNAs. In this process, shRNA expressed by the Salmonella silences or knocks down genes of interest (target genes) inside target cells. The nucleic acid expression constructs of the invention include an RNA polymerase (e.g., a T7 polymerase), an RNA polymerase promoter (e.g., a T7 polymerase promoter), and an RNA polymerase terminator (e.g., a T7 polymerase terminator). The Salmonella bacteria can also include, on the same or different nucleic acid construct, an endosomal release factor (e.g., HlyA).

### CROSS-REFERENCE TO RELATED APPLICATIONS

This application claims benefit of U.S. Provisional Application No. 61/233,630, filed Aug. 13, 2009, which is hereby incorporated by reference.

### BACKGROUND OF THE INVENTION

The invention relates to the inhibition of gene expression in host cells by administering bacteria expressing shRNA constructs.

Gene silencing through RNAi (RNA-interference) by use of short interfering RNA (siRNA) has emerged as a powerful tool for molecular biology and holds the potential to be used for therapeutic gene silencing. Short hairpin RNA (shRNA) transcribed from small DNA plasmids within the target cell has also been shown to mediate stable gene silencing and achieve gene knockdown at levels comparable to those obtained by transfection with chemically synthesized siRNA.

Possible applications of RNAi for therapeutic purposes are extensive and include silencing and knockdown of disease genes such as oncogenes or viral genes. One major obstacle for the therapeutic use of RNAi is the delivery of siRNA to the target cell.

### SUMMARY OF THE INVENTION

In one aspect, the invention features a method of reducing the expression of a target gene in a cell (e.g., a tumor cell or an intestinal cell) by contacting the cell with live bacteria from the genus Salmonella (e.g., Salmonella typhimurium, or more specifically, Salmonella typhimurium aroA 7207), wherein the bacteria includes nucleic acid sequences encoding a T7 polymerase and a T7 expression cassette, wherein the T7 expression cassette includes a T7 promoter, a T7 terminator, and a nucleic acid sequence encoding an shRNA construct corresponding to the target gene.

In another aspect, the invention features a nucleic acid molecule (e.g., a pharmaceutical composition) encoding a T7 polymerase, an HlyA gene, and a T7 expression cassette, wherein the T7 expression cassette includes a T7 promoter, a T7 terminator, and a nucleic acid sequence encoding an shRNA (e.g., an shRNA corresponding to a target gene). The invention also features bacteria from the genus Salmonella (e.g., Salmonella typhimurium, or more specifically, Salmonella typhimurium aroA 7207) including any of the foregoing nucleic acid molecules.

In any of the foregoing aspects, the nucleic acid sequence can also include an endosomal release factor (e.g., HlyA).

Also in any of the foregoing aspects, the target gene can be, for example, ABL1, β-catenin, BCL1, BCL2, BCL6, CBFA2, CBL, CSF1R, ERBA, ERBB, EBRB2, ETS1, ETS1, ETV6, FOR, FOS, FYN, HCR, HRAS, JUN, KRAS, LCK, LYN, MDM2, MLL, MYB, MYC, MYCL1, MYCN, NRAS, PIM1, PML, RET, SRC, TALI, TCL3, and YES.

Any of the above cells can be found, e.g., in a human (e.g., a human with cancer, an inflammatory disorder, and/or a bacterial or viral infection). Any of the above bacteria can be administered (e.g., to a human) orally or intravenously.

By “target gene” is meant the particular genetic unit that exhibits reduced expression by the introduction of shRNA. The term “target gene” is meant to include wild type and mutant genes endogenous to the cell type or organism that is contacted with the bacteria of the invention as well as non-endogenous genes (e.g., genes introduced by viruses or other pathogens).

By “shRNA” is meant RNA comprising a duplex region complementary to an mRNA. For example, a short hairpin RNA (shRNA) may comprise a duplex region containing nucleoside bases, where the duplex is between 17 and 29 bases in length, and the strands are separated by a single-stranded 4, 5, 6, 7, 8, 9, or 10 base linker region. Optimally, the linker region is 6 bases in length.

By “shRNA construct corresponding to said target gene” is meant an shRNA construct which contains sufficient complementarity to the target gene to achieve reduced expression.

By “reducing the expression” of a target gene is meant a reduction in protein or nucleic acid level or activity in a cell, a cell extract, or a cell supernatant. For example, such a decrease may be due to reduced RNA stability, transcription, or translation, or RNA interference. Preferably, this decrease is at least 5%, 10%, 25%, 50%, 75%, 80%, or even 90% of the level of expression under control conditions.

By “an shRNA construct corresponding to said target gene” is meant an shRNA molecule that specifically hybridizes to a target gene. By “specifically hybridizes” is meant an shRNA that hybridizes to a target nucleic acid molecule but does not substantially hybridize to other nucleic acid molecules in a sample (e.g., a sample from a cell) that naturally includes the target gene, when assayed under denaturing conditions. In one embodiment, the amount of a target nucleic acid molecule hybridized to, or associated with, the shRNA, as measured using standard assays, is 2-fold, desirably 5-fold, more desirably 10-fold, and most desirably 50-fold greater than the amount of a control nucleic acid molecule hybridized to, or associated with, the shRNA.

As used herein, an “endosomal release factor” is a factor, e.g., a protein or a group of proteins which, when expressed by a bacteria, allow escape of the shRNA from the endosome into the cytosol of the target cell after the carrier bacteria is ruptured.

By “substantially identical” is meant a nucleic acid or amino acid sequence that, when optimally aligned, for example using the methods described below, share at least 60%, 65%, 70%, 75%, 80%, 85%, 90%, 95%, 96%, 97%, 98%, 99%, or 100% sequence identity with a second nucleic acid or amino acid sequence. “Substantial identity” may be used to refer to various types and lengths of sequence, such as full-length sequence, functional domains, coding and/or regulatory sequences, exons, introns, promoters, and genomic sequences. Percent sequence identity between two polypeptides or nucleic acid sequences is determined in various ways that are within the skill in the art, for instance, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed pp 353-358; BLAST program (Basic Local Alignment Search Tool; (Altschul, S. F., W. Gish, et al. (1990) J Mol Biol 215: 403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. It is understood that for the purposes of determining sequence identity when comparing a DNA sequence to an RNA sequence, a thymine nucleotide is equivalent to a uracil nucleotide. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

Other features and advantages of the invention will be apparent from the following Detailed Description, the drawings, and the claims.

### BRIEF DESCRIPTION OF THE DRAWINGS

FIG. 1 is a diagram showing the nucleic acid organization of the TRIPIII-CAT plasmid.

FIG. 2A is a Western blog showing expression of T7 RNA polymerase in SL-7207 transformed with TRIPIII-CAT plasmid using mouse monoclonal antibody (mAb) against T7 RNA polymerase. Lanes 1-4, four colonies of SL-TRIPIII-CAT; lane 5, negative control from SL.

FIG. 2B is a Western blot showing expression of HlyA in SL-7207 transformed with TRIPIII-CAT plasmid using mouse monoclonal antibody against HlyA. Lanes 1-4, SL-TRIP III-CAT from four different colonies; lane 5, negative control from untransformed SL7207.

FIG. 3 is a graph showing shRNA expression levels in attenuated Salmonella 7207 (SL) as measured by QRT-PCR. The results show that SL-TRIPIII-CAT colonies do not give consistent shRNA levels from different colonies but do show a similar concentration as the BL21 (DE3)-TRIPI system.

FIG. 4A is a Western Blot showing protein levels of β-catenin and β-actin in treated SW480 cell line. Significant silencing of β-catenin is observed atn different MOI. Lanes 1 and 2, blank control without treatment; lanes 3 and 4, treatment control with blank SL containing no plasmid at MOI 1:2000; lanes 5 and 6, SW480 treated with SL-TRIPIII-CAT at MOI 1:2000; lanes 7 and 8, SW480 treated with SL-TRIPIII-CAT at MOI 1:4000.

FIG. 4B is a graph showing mRNA expression levels relative to β-actin after treatment of SW480 with SL-TRIPIII-CAT at MOI 1:2000, 1:4000, and 1:8000 as measured by QRT-PCR; two controls were set up in MOI 1:8000, including SL-TRIPIII-TAT and SL7207 (no plasmid).

FIG. 5A is a Western Blot showing a comparison of gene silencing between siRNA transfection or treatment with TRIPIIIΔHlyA-CAT or TRIPIII-CAT. To compare the efficiency of gene silencing, SW480 cells were treated with SL7207 containing TRIPIIIΔHlyA or TRIP III targeting β-catenin (SL-TRIPIIIΔHlyA-CAT and SL-TRIPIII-CAT) or siRNA of the same target sequence. Lanes 1 and 2, SL7207 only (no plasmid) treatment at MOI 1:4000; lanes 3 and 4, transfection (Lipofectamine-2000) with β-catenin siRNA (20 nM); lanes 5 and 6, SL-TRIPIIIΔHlyA-CAT; lanes 7 and 8, SL-TRIPIII-CAT.

FIG. 5B is a Western Blot showing detection β-catenin expression in SW480 cells (48 hr) with different bacterial treatments at MOI 1:4000. Lanes 1 and 2, untreated control; lanes 3 and 4, SL (no plasmid) treatment with MOI 1:4000; lanes 5 and 6, SL-TRIPIII-TAT (SL7207 with TRIP III containing shRNA sequence against HIV TAT protein), MOI 1:4000; lanes 7 and 8, E. coli (BL21DE3) with TRIP I-CAT, MOI 1:4000; lanes 9 and 10, SL-TRIPIII-CAT, MOI 1:4000.

FIGS. 6A-6C are graphs showing Annexin-V staining as a function of propidium iodide staining as a result of flow cytometry. The treated cells were collected after 48 hr. the Y axis shows the level of necrosis; the X axis shows the level of apoptosis. Cells in lower left quadrant are considered alive/healthy. FIG. 6A, cells without treatment; FIG. 6B, cells treated with EB-TRIPI; FIG. 6C, cells treated with SL-TRIPIII.

FIG. 7 is a graph showing cell proliferation rate. SL-TRIPIII-CAT can inhibit the growth of SW480 by 80% while SL and SL-TRIPIII-TAT have 60% suppression of SW480 growth.

FIG. 8A is a series of photomicrographs showing EB-TRIPI invasion of SW480 cells. a. bacteria have already entered cells but have not been lysed and cells are still normal; b. few invading bacteria have been lysed and cell nucleolus has become condensed; c. recovered cells are not completely healthy and most bacteria remain stuck to the surface of nucleolus. d-h show an experiment where more bacteria enter a target cell: d. bacteria entered cell and a few of them have been lysed; e. the whole cell body has shrunk and the nucleolus has become condensed and most bacteria remain stuck to the surface of nucleolus; f. the cytoplasm of the cell has almost disappeared and nucleolus has become more condensed; g. the nucleolus has become even more condensed and the cytoplasm totally disappeared; h. shows that too many bacteria can directly lyse or disrupt the target cell.

FIG. 8B is a series of photomicrographs showing SL-TRIPIII invasion of SW480. a. normal cell before invasion; b. bacteria entered cells but have not been lysed and nucleolus has become condensed; c. all of invaded bacteria have lysed and lysed mixture approached the nucleolus; d. the lysed mixture has been digested and the cell is recovering; e, when the lysed mixture was totally digested, the cell has fully recovered.

FIG. 9A is a graph showing tumor size as a function of time in three groups treated with the indicated construct. 33 nude mice with SW480 xenograft tumors were randomized into three groups. PBS (n=10), SL-TRIPIII-TAT (n=10), SL-TRIPIII-CAT (n=13). Animals were treated with intravenous injections of 10⁶ colony forming units (cfu) of SL-TRIPIII bacteria in 100 μl or equal volume of PBS every other day for 2 weeks. During the in-life phase of the study, tumor sizes were measured using electronic caliper. The graph shows the treatment with SL-TRIPIII-CAT leads to significant reduction in tumor growth compared with those treated with SL-TRIPIII-TAT (p=0.02<0.05) or with PBS (p=0.009<0.01).

FIG. 9B is a graph showing the distribution of tumor weight after the animals were sacrificed by treatment group. The graph shows that the tumor weights from those treated with SL-TRIPIII-CAT were significantly lighter than those treated with PBS or SL-TRIPIII-TAT.

FIG. 9C is a Western blot showing the concentration of the indicated protein in tumors selected from the indicated treatment groups.

FIG. 9D is a series of photomicrographs showing immunocytohistological staining of β-catenin (CTNNB1) in tumor sections. a and d, tumor sections from PBS treatment group; b and e, tumor sections from SL-TRIPIII-TAT treatment group; c and f, tumor sections from SL-TRIPIII-CAT treatment group.

FIG. 10 is a Western blot showing expression of the indicated proteins in samples isolated from mice in the indicated treatment groups. ApcMin mice were treated with SL-TRIPIII via oral administration. 19 ApcMin mice were randomized into three groups and treated with PBS (n=6), SL-TRIPIII-TAT (n=7), and SL-TRIPIII-PMC2 (n=6). Animals were treated with oral administration of 10⁸ colony forming units (cfu) of SLTRIPIII bacteria in 100 ul or equal volume of PBS every other day for 2 weeks. Animals were sacrificed and polyps were harvested from the intestines. Western blot analysis shows very similar results to those from the xenograft cancer model. The protein level of β-catenin is knocked down by 70% and 50% compared with those in PBS treatment and SL-TRIPIII-TAT treatment.

### DETAILED DESCRIPTION

The invention features compositions and methods for delivering small interfering (siRNAs), e.g., shRNAs, to host cells using non-pathogenic strains of Salmonella bacteria containing nucleic acid expression constructs encoding shRNAs. This process is herein referred to as transkingom RNAi or tkRNAi. In this process, shRNA expressed by the Salmonella silences or knocks down genes of interest (target genes) inside target cells. The nucleic acid expression constructs of the invention include an RNA polymerase (e.g., a T7 polymerase), an RNA polymerase promoter (e.g., a T7 polymerase promoter), and an RNA polymerase terminator (e.g., a T7 polymerase terminator). The Salmonella bacteria can also include, on the same or different nucleic acid construct, an endosomal release factor (e.g., HlyA).

Bacterial delivery of shRNA is more attractive than viral delivery as it can be controlled by use of antibiotics and attenuated bacterial strains that are unable to multiply. Also, bacteria are much more accessible to genetic manipulation which allows the production of vector strains specifically tailored to certain applications.

Liberation of shRNA encoding plasmid from the intracellular bacteria occurs through active mechanisms. One mechanism involves the type III export system in S. typhimuriumrn, a specialised multiprotein complex spanning the bacterial cell membrane whose functions include secretion of virulence factors to the outside of the cell to allow signaling towards the target cell, but which can also be used to deliver antigens into target cells or through bacterial lysis and liberation of bacterial contents into the cytoplasm. The lysis of intracellular bacteria is triggered through addition of an intracellularly active antibiotic (tetracycline) or occurs naturally through bacterial metabolic attenuation (auxotrophy). After liberation of the eukaryotic transcription plasmid, shRNA or siRNA are produced within the target cell and trigger the highly specific process of mRNA degradation, which results in silencing of the targeted gene.

The non-virulent bacteria of the invention have invasive properties and may enter a mammalian host cell through various mechanisms. In contrast to uptake of bacteria by professional phagocytes, which normally results in the destruction of the bacterium within a specialized lysosome, invasive bacteria strains have the ability to invade non-phagocytic host cells.

The tkRNAi methods of the invention are used to create transient “knockdown” genetic animal models as opposed to genetically engineered knockout models to discover gene functions. The methods are also used as in vitro transfection tool for research and drug development.

The tkRNAi methods of the invention are used for delivery of gene silencing to the gut and colon, and for oral application in the treatment of various diseases, namely colon cancer treatment and prevention. In another aspect of this embodiment, delivery of gene silencing is extra-intestinal.

### Bacterial Delivery of RNA to Eukaryotic Cells

Delivery of at least one molecule into a target cell can be determined according to methods known in the art. For example, the presence of the molecule, by the decrease in expression of an RNA or protein silenced thereby, can be detected by hybridization or PCR methods, or by immunological methods that may include the use of an antibody. Determining whether a microorganism is sufficiently invasive for use in the invention may include determining whether sufficient RNA was delivered to host cells relative to the number of microorganisms contacted with the host cells. If the amount of RNA is low relative to the number of microorganisms used, it may be desirable to further modify the microorganism to increase its invasive potential.

Bacterial entry into cells can be measured by various methods. Intracellular bacteria survive treatment by aminoglycoside antibiotics, whereas extracellular bacteria are rapidly killed. A quantitative estimate of bacterial uptake can be achieved by treating cell monolayers with the antibiotic gentamicin to inactivate extracellular bacteria, then by removing the antibiotic before liberating the surviving intracellular organisms with gentle detergent and determining viable counts on standard bacteriological medium. Furthermore, bacterial entry into cells can be directly observed, e.g., by thin-section-transmission electron microscopy of cell layers or by immunofluorescent techniques Thus, various techniques can be used to determine whether a specific bacteria is capable of invading a specific type of cell or to confirm bacterial invasion following modification of the bacteria, such modification of the tropism of the bacteria to mimic that of a second bacterium. Bacteria that can be used for delivering RNA according to the method of the invention are preferably non-pathogenic. However, pathogenic bacteria can also be used, so long as their pathogenicity has been attenuated to thereby render the bacteria non-harmful to a subject to which it is administered. As used herein, the term “attenuated bacterium” refers to a bacterium that has been modified to significantly reduce or eliminate its harmfulness to a subject.

Examples of Salmonella strains which can be employed in the present invention include Salmonella typhi (ATCC No. 7251) and S. typhimurium (ATCC No. 13311). Attenuated Salmonella strains are preferably used in the present invention and include S. typhi-aroC-aroD (Hone et al. Vacc. 9:810 (1991) S. typhimurium-aroA mutant (Mastroeni et al. Micro. Pathol. 13:477 (1992)) and Salmonella typhimurium 7207. Alternatively, new attenuated Salmonella strains can be constructed by introducing one or more attenuating mutations as described below. Furthermore, the invention also features the use of non-Salmonella organisms for the delivery of shRNA (e.g., malaria parasite, microplasma, Cryptococuus neoformans, and Brucellosis. In these organisms, the T7 polymerase can be under the control promoter which functions in the respective organism. Furthermore, any other invasive bacteria can be used in the methods of the invention, including strains of Shigella, Mycobacteria, Mycoplasma, and Listeria.

Attenuating mutations can be introduced into bacterial pathogens using non-specific mutagenesis either chemically, using agents such as N-methyl-N′-nitro-N-nitrosoguanidine, or using recombinant DNA techniques; classic genetic techniques, such as Tn10 mutagenesis, P22-mediated transduction, λ phage mediated crossover, and conjugational transfer; or site-directed mutagenesis using recombinant DNA techniques. Recombinant DNA techniques are preferable since strains constructed by recombinant DNA techniques are far more defined. Examples of such attenuating mutations include, but are not limited to:

(i) auxotrophic mutations, such as aro (Hoiseth et al. Nature, 291:238-239 (1981)), gua (McFarland et al Microbiol. Path., 3:129-141 (1987)), nad (Park et al. J. Bact, 170:3725-3730 (1988), thy (Nnalue et al. Infect. Immun., 55:955-962 (1987)), and asd (Curtiss, supra) mutations;

(ii) mutations that inactivate global regulatory functions, such as cya (Curtiss et al. Infect. Immun., 55:3035-3043 (1987)), crp (Curtiss et al (1987), supra), phoP/phoQ (Groisman et al. Proc. Natl. Acad. Sci., USA, 86:7077-7081 (1989); and Miller et al. Proc. Natl. Acad. Sci., USA, 86:5054-5058 (1989)), phop^{c} (Miller et al. J. Bact, 172:2485-2490 (1990)) or ompR (Dorman et al. Infect. Immun., 57:2136-2140 (1989)) mutations;

(iii) mutations that modify the stress response, such as recA (Buchmeier et al. MoI. Micro., 7:933-936 (1993)), htrA (Johnson et al. MoI. Micro., 5:401-407 (1991)), htpR (Neidhardt et al. Biochem. Biophys. Res. Com., 100:894-900 (1981)), hsp (Neidhardt et al. Ann. Rev. Genet, 18:295-329 (1984)) and groEL (Buchmeier et al. Sci., 248:730-732 (1990)) mutations;

(iv) mutations in specific virulence factors, such as IsyA (Libby et al. Proc. Natl. Acad. Sci., USA, 91:489-493 (1994)), pag or prg (Miller et al (1990), supra; and Miller et al (1989), supra), iscA or virG (d'Hauteville et al. MoI. Micro., 6:833-841 (1992)), plcA (Mengaud et al. Mol. Microbiol., 5:367-72 (1991); Camilli et al. J. Exp. Med, 173:751-754 (1991)), and act (Brundage et al. Proc. Natl. Acad. Sci., USA, 90:11890-11894 mutations;

(v) mutations that affect DNA topology, such as top A (Galan et al. Infect. Immun., 58: 1879-1885 (1990));

(vi) mutations that disrupt or modify the cell cycle, such as min (de Boer et al. Cell, 56:641-649 (1989)).

(vii) introduction of a gene encoding a suicide system, such as sacB (Recorbet et al. App. Environ. Micro., 59:1361-1366 (1993); Quandt et al. Gene, 127:15-21 (1993)), nuc (Ahrenholtz et al. App. Environ. Micro., 60:3746-3751 (1994)), hok, gef, kil, or phlA (Molin et al. Ann. Rev. Microbiol., 47:139-166 (1993));

(viii) mutations that alter the biogenesis of lipopolysaccharide and/or lipid A, such as rFb (Raetz in Esherishia coli and Salmonella typhimurium, Neidhardt et al, Ed., ASM Press, Washington D.C. pp 1035-1063 (1996)), galE (Hone et al. J. Infect. Dis., 156:164-167 (1987)) and htrB (Raetz, supra), msbB (Reatz, supra); and

(ix) introduction of a bacteriophage lysis system, such as lysogens encoded by P22 (Rennell et al. Virol, 143:280-289 (1985)), λ murein transglycosylase (Bienkowska-Szewczyk et al. Mol. Gen. Genet., 184:111-114 (1981)) or S-gene (Reader et al. Virol, 43:623-628 (1971)).

The attenuating mutations can be either constitutively expressed or under the control of inducible promoters, such as the temperature sensitive heat shock family of promoters (Neidhardt et al. supra), or the anaerobically induced nirB promoter (Harbome et al. MoL Micro., 6:2805-2813 (1992)) or repressible promoters, such as uapA (Gorfinkiel et al. J. Biol. Chem., 268:23376-23381 (1993)) or gcv (Stauffer et al. J. Bact, 176:6159-6164 (1994)).

### Target Cells

The invention provides a method for delivering RNA to any type of target cell. As used herein, the term “target cell” refers to a cell which can be invaded by a bacterium, i.e., a cell which has the necessary surface receptor for recognition by the bacterium.

Preferred target cells are eukaryotic cells. Even more preferred target cells are animal cells. “Animal cells” are defined as nucleated, non-chloroplast containing cells derived from or present in multicellular organisms whose taxanomic position lies within the kingdom animalia. The cells may be present in the intact animal, a primary cell culture, explant culture or a transformed cell line. The particular tissue source of the cells is not critical to the present invention. The recipient animal cells employed in the present invention are not critical thereto and include cells present in or derived from all organisms within the kingdom animalia, such as those of the families mammalia, pisces, avian, and reptilia.

Preferred animal cells are mammalian cells, such as humans, bovine, ovine, porcine, feline, canine, goat, equine, murine, rodent, and primate cells. The most preferred animal cells are human cells.

In a preferred embodiment, the target cell is in a mucosal surface. Salmonella are naturally adapted for this application as these organisms possess the ability to attach to and invade host mucosal surfaces. Therefore such bacteria can deliver RNA molecules or RNA-encoding DNA to cells in the host mucosal compartment.

Although certain types of bacteria may have a certain tropism, i.e., preferred target cells, delivery of RNA or RNA-encoding DNA to a certain type of cell can be achieved by choosing a bacterium which has a tropism for the desired cell type or which is modified such as to be able to invade the desired cell type.

Bacteria can also be targeted to other types of cells. For example, bacteria can be targeted to erythrocytes of humans and primates by modifying bacteria to express on their surface either, or both of, the Plasmodium vivax reticulocyte binding proteins-1 and -2, which bind specifically to erythrocytes in humans and primates (Galinski et al. Cell, 69: 1213-1226 (1992)). In another embodiment, bacteria are modified to have on their surface asialoorosomucoid, which is a ligand for the asilogycoprotein receptor on hepatocytes (Wu et al. J. Biol. Chem., 263:14621-14624 (1988)). In yet another embodiment, bacteria are coated with insulin-poly-L-lysine, which has been shown to target plasmid uptake to cells with an insulin receptor (Rosenkranz et al. Expt. Cell Res., 199:323-329 (1992)). Also within the scope of the invention are bacteria modified to have on their surface p60 of Listeria monocytogenes, which allows for tropism for hepatocytes (Hess et al. Infect. Immun., 63:2047-2053 (1995)), or a 60 kD surface protein from Trypanosoma cruzi which causes specific binding to the mammalian extra-cellular matrix by binding to heparin, heparin sulfate and collagen (Ortega-Barria et al. Cell, 67:411-421 (1991)).

Yet in another embodiment, a cell can be modified to become a target cell of a bacterium for delivery of RNA. Accordingly, a cell can be modified to express a surface antigen which is recognized by a bacterium for its entry into the cell, i.e., a receptor of an invasion factor. The cell can be modified either by introducing into the cell a nucleic acid encoding a receptor of an invasion factor, such that the surface antigen is expressed in the desired conditions. Alternatively, the cell can be coated with a receptor of an invasion factor. Receptors of invasion factors include proteins belonging to the integrin receptor superfamily. A list of the type of integrin receptors recognized by various bacteria and other microorganisms can be found, e.g., in Isberg and Tran Van Nliieu (1994) Ann. Rev. Genet. 27:395. Nucleotide sequences for the integrin subunits can be found, e.g., in GenBank, which is publicly available on the internet.

Examples of bacteria which can naturally access the cytoplasm of avian cells include, but are not restricted to, Salmonella galinarum (ATCC No. 9184), Salmonella enteriditis (ATCC No. 4931) and Salmonella typhimurium (ATCC No. 6994). Attenuated bacteria are preferred to the invention and include attenuated Salmonella strains such as S. galinarum cya crp mutant (Curtiss et al. (1987) supra) or S. enteritidis aroA aromatic-dependent mutant CVL30 (Cooper et al. Infect. Immun., 62:4739-4746 (1994)).

Examples of bacteria which can naturally access the cytoplasm of reptilian cells include, but are not restricted to, Salmonella typhimurium (ATCC No. 6994). Attenuated bacteria are preferable to the invention and include, attenuated strains such as S. typhimuirum aromatic-dependent mutant (Hormaeche et al. supra).

Set forth below are examples of cell lines to which RNA can be delivered according to the method of this invention.

Examples of human cell lines include but are not limited to ATCC Nos. CCL 62, CCL 159, HTB 151, HTB 22, CCL 2, CRL 1634, CRL 8155, HTB 61, and HTB104.

Examples of bovine cell lines include ATCC Nos. CRL 6021, CRL 1733, CRL 6033, CRL 6023, CCL 44 and CRL 1390. Examples of ovine cells lines include ATCC Nos. CRL 6540, CRL 6538, CRL 6548 and CRL 6546.

Examples of porcine cell lines include ATCC Nos. CL 184, CRL 6492, and CRL 1746.

Examples of feline cell lines include CRL 6077, CRL 6113, CRL 6140, CRL 6164, CCL 94, CCL 150, CRL 6075 and CRL 6123.

Examples of buffalo cell lines include CCL 40 and CRL 6072.

Examples of canine cells include ATCC Nos. CRL 6213, CCL 34, CRL 6202, CRL 6225, CRL 6215, CRL 6203 and CRL 6575.

Examples of goat derived cell lines include ATCC No. CCL 73 and ATCC No. CRL 6270.

Examples of horse derived cell lines include ATCC Nos. CCL 57 and CRL 6583.

Examples of deer cell lines include ATCC Nos. CRL 6193-6196.

Examples of primate derived cell lines include those from chimpanzee's such as ATCC Nos. CRL 6312, CRL 6304, and CRL 1868; monkey cell lines such as ATCC Nos. CRL 1576, CCL 26, and CCL 161; orangutan cell line ATCC No. CRL 1850; and gorilla cell line ATCC No. CRL 1854.

### Pharmaceutical Compositions

In a preferred embodiment of the invention, the invasive bacteria containing the RNA molecules, and/or DNA encoding such, are introduced into an animal by intravenous, intramuscular, intradermal, intraperitoneally, peroral, intranasal, intraocular, intrarectal, intravaginal, intraosseous, oral, immersion and intraurethral inoculation routes.

The amount of the live invasive bacteria of the present invention to be administered to a subject will vary depending on the species of the subject, as well as the disease or condition that is being treated. Generally, the dosage employed will be about 10³ to 10¹¹ viable organisms, preferably about 10⁵ to 10⁹ viable organisms per dose.

The invasive bacteria of the present invention are generally administered along with a pharmaceutically acceptable carrier and/or diluent. The particular pharmaceutically acceptable carrier an/or diluent employed is not critical to the present invention. Examples of diluents include a phosphate buffered saline, buffer for buffering against gastric acid in the stomach, such as citrate buffer (pH 7.0) containing sucrose, bicarbonate buffer (pH 7.0) alone (Levine et al. J. Clin. Invest, 79:888-902 (1987); and Black et al J. Infect. Dis., 155:1260-1265 (1987)), or bicarbonate buffer (pH 7.0) containing ascorbic acid, lactose, and optionally aspartame (Levine et al. Lancet, 11:467-470 (1988)). Examples of carriers include proteins, e.g., as found in skim milk, sugars, e.g., sucrose, or polyvinylpyrrolidone. Typically these carriers would be used at a concentration of about 0.1-30% (w/v) but preferably at a range of 1-10% (w/v).

Set forth below are other pharmaceutically acceptable carriers or diluents which may be used for delivery specific routes. Any such carrier or diluent can be used for administration of the bacteria of the invention, so long as the bacteria are still capable of invading a target cell. In vitro or in vivo tests for invasiveness can be performed to determine appropriate diluents and carriers. The compositions of the invention can be formulated for a variety of types of administration, including systemic and topical or localized administration. Lyophilized forms are also included, so long as the bacteria are invasive upon contact with a target cell or upon administration to the subject. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the composition, e.g., bacteria, of the invention can be formulated in liquid solutions, preferably in physiologically compatible buffers such as Hank's solution or Ringer's solution.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

The pharmaceutical compositions may be formulated for parenteral administration by injection, e.g., by bolus injection or continuous infusion. Formulations for injection may be presented in unit dosage form, e.g., in ampoules or in multi-dose containers, with an added preservative.

The pharmaceutical compositions may also be formulated in rectal, intravaginal, or intraurethral compositions such as suppositories or retention enemas.

For topical administration, the bacteria of the invention are formulated into ointments, salves, gels, or creams as generally known in the art, so long as the bacteria are still invasive upon contact with a target cell.

The compositions may, if desired, be presented in a pack or dispenser device and/or a kit which may contain one or more unit dosage forms containing the active ingredient. The pack may for example comprise metal or plastic foil, such as a blister pack. The pack or dispenser device may be accompanied by instructions for administration.

The invasive bacteria containing the RNA-encoding DNA to be introduced can be used to infect animal cells that are cultured in vitro, such as cells obtained from a subject. These in vitro-infected cells can then be introduced into animals, e.g., the subject from which the cells were obtained initially, intravenously, intramuscularly, intradermally, or intraperitoneally, or by any inoculation route that allows the cells to enter the host tissue. When delivering RNA to individual cells, the dosage of viable organisms to administered will be at a multiplicity of infection ranging from about 0.1 to 10⁶, preferably about 10² to 10⁴ bacteria per cell.

### Target Genes

The tkRNAi methods of the invention can be used to treat any disease whereby reduction in the expression of a particular gene is desired.

The tkRNAi methods of the invention are useful as a cancer therapy or to prevent cancer. This method is effected by silencing or knocking down genes involved with cell proliferation or other cancer phenotypes. Examples of these genes are k-Ras and β-catenin. Specifically, k-Ras and β-catenin are targets for RNAi based therapy of colon cancer. These oncogenes are active and relevant in the majority of clinical cases. Bacteria is administered to reach the intestinal tract for colon cancer treatment and prevention. These methods are also used to treat animals carrying xenograft tumors, to treat and prevent cancer in k-RasV12 model of intestinal tumorgenesis, and to prevent and treat tumors in the adenomatous polyposis coli min mouse model (APC-min model). In this model, the mouse has a defective APC gene resulting in the formation of numerous intestinal and colonic polyps which is used as an animal model for human familiar adenomatous polyposis coli (FAP) of intestinal tumorigenesis.

Additional examples of cancer that can be treated using the tkRNAi methods of the invention include leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

The invention features the following classes of possible tkRNAi target genes: developmental genes (adhesion molecules, cyclin kinase inhibitors, Wnt family members, Pax family members, Winged helix family members, Hox family members, cytokines/lymphok-ines and their receptors, growth/differentiation factors and their receptors, angiogenic factors and their receptors such as VEGF, HIF, VEGFR, antiangiogenic factors and their receptors, neurotransmitters and their receptors); oncogenes (e.g., ABL1, BCL1, BCL2, BCL6, CBFA2, CBL, CSF1R, ERBA, ERBB, EBRB2, ETS1, ETS1, ETV6, FOR, FOS, FYN, HCR, HRAS, JUN, KRAS, LCK, LYN, MDM2, MLL, MYB, MYC, MYCL1, MYCN, NRAS, PIM1, PML, RET, SRC, TALI, TCL3, and YES); tumor suppressor genes (e.g., APC, BRCA1, BRCA2, MADH4, MCC, NF1, NF2, RB1, TP53, and WT1); and enzymes (e.g., ACC synthases and oxidases, ACP desaturases and hydroxylases, ADP-glucose pyrophorylases, ATPases, alcohol dehydrogenases, amylases, amyloglucosidases, catalases, cellulases, chal-cone synthases, chitinases, cyclooxygenases, decarboxy-lases, dextrinases, DNA and RNA polymerases, galactosi-dases, glucanases, glucose oxidases, granule-bound starch synthases, GTPases, helicases, hemicellulases, integrases, inulinases, invertases, isomerases, kinases, lactases, lipases, lipoxygenases, lysozymes, nopaline synthases, octopine synthases, pectinesterases, peroxidases, phosphatases, phos-pholipases, phosphorylases, phytases, plant growth regulator synthases, polygalacturonases, proteinases and pepti-dases, pullanases, recombinases, reverse transcriptases, topoisomerases, and xylanases).

The nucleic acid constructs of the invention can be used to screen different cancer-related targets in transgenic as well as wild type animals for therapeutic experiments. The tkRNAi methods of the invention are also used to treat or prevent viral diseases (e.g. hepatitis) and genetic disorders.

The tkRNAi methods of the invention are also used to create cancer-preventing “probiotic bacteria” for use, especially with the target of GI tract or liver.

The tkRNAi methods of the invention are used as therapy against inflammatory conditions, e.g. hepatitis, inflammatory bowel disease (IBD) or colitis. These methods are used to silence or knockdown non-cancer gene targets (viral genes, for treatment and prevention of hepatitis B, C; inflammatory genes, for treatment and prevention of inflammatory bowel disease) and others.

### EXAMPLES

In the following example, transkindom interference plasmid III (TRIPIII) was constructed and transformed into attenuated Salmonella typhimurium 7207 (SL). SL produced, delivered and released shRNA into host cells. The results show 100% silencing of β-catenin at the protein level. Nude mice carrying SW480 xenograft tumors were treated with SL containing TRIPIII (SL-TRIPIII), in which the expression level of β-catenin and its regulated genes c-Myc and cyclin D1 were decreased respectively by 50%, 40%, and 30% in tumors. The results further show that ApcMin mice treated with SL-TRIPIII show the reduction of those three genes by 50%, 25% and 40% in polyps respectively. Previous studies regarding tkRNAi were disclosed in, for example, PCT Application Publication No. WO 2006/066048, which is hereby incorporated by reference in its entirety.

Plasmid Construction

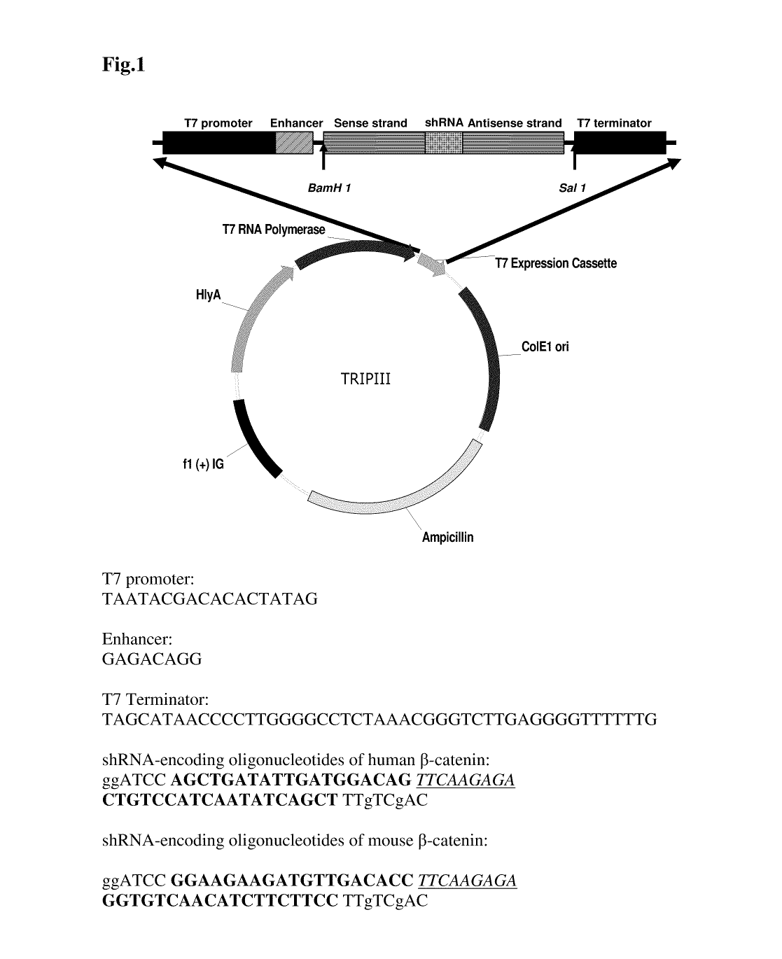

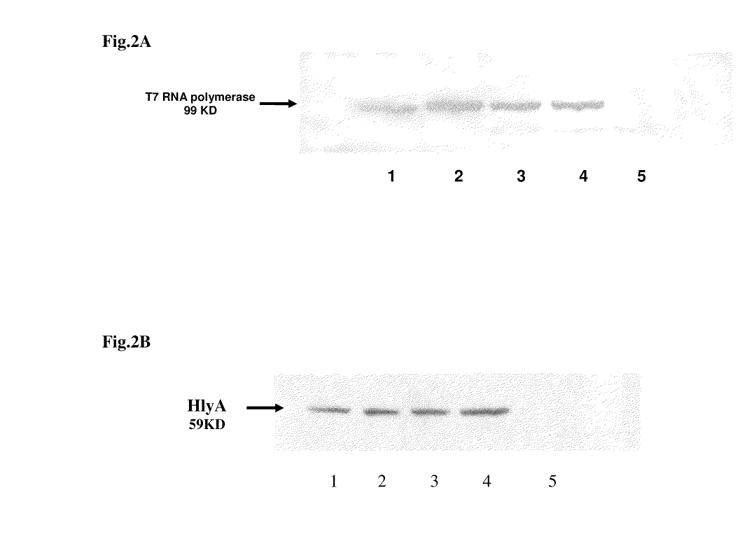

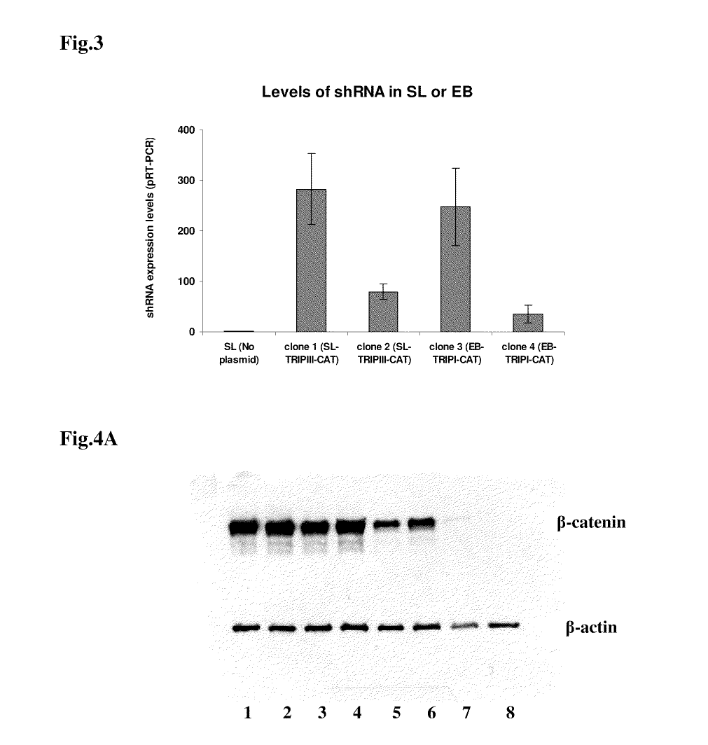

To establish a plasmid-based system used in attenuated Salmonella, TRIPIII including HlyA gene, T7 RNA polymerase gene, T7 promoter driven expression cassette was constructed and the map of Transkindom Interference Plasmid III (TRIPIII) as shown in FIG. 1. FIGS. 2A and 2B show the expression of both HlyA and T7 RNA polymerase in attenuated Salmonella. Oligonucleotides encoding shRNA against HIV TAT and human or mouse β-catenin (CAT and PMC2) were put into the multi-cloning site (MCS) of TRIPIII to make the TRIPIII-TAT, TRIPIII-CAT, or TRIPIII-PMC2 and then transformed into attenuated Salmonella to make the strains of SL-TRIPIII-TAT, CAT, and PMC2. The positive colonies were selected according to the expression level of shRNA by QRT-PCR. FIG. 3 shows the shRNA levels from different positive SL colonies.

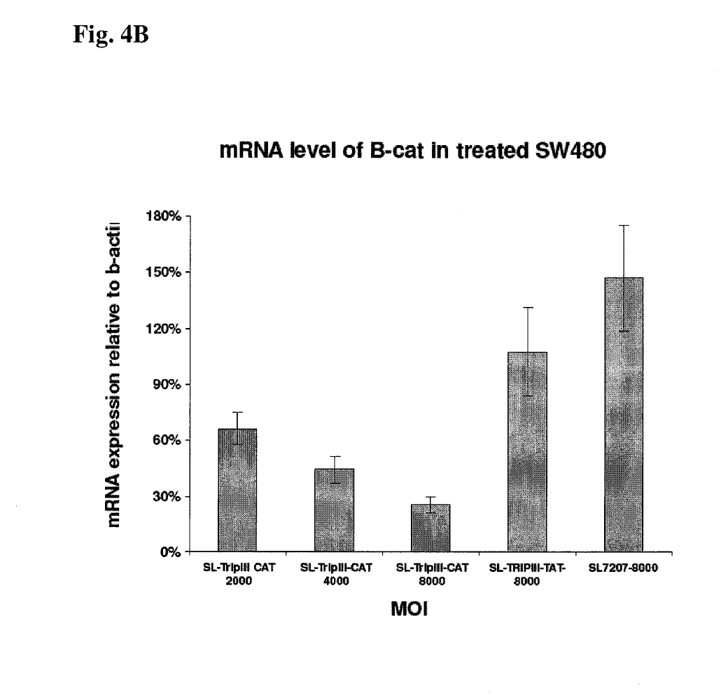

Silencing β-Catenin Expression by SL-TRIPIII-CAT SW480 cells were treated for 2 hr at different MOI with SL-TRIPIII-CAT which was confirmed to have high levels of shRNA against β-catenin. Cells were harvested after 48 hr. Total protein from treated SW480 was purified and run with PAGE Gel. FIGS. 4A-4B show the protein level of β-catenin in treated SW480. Comparing to the controls without treatment, SL did not decrease β-catenin levels while SL-TRIPIII-CAT reduced β-catenin protein levels at MOIs of 1:2000 and 1:4000 and showed gene silencing by 50% in the former and almost 100% in the latter. Moreover, quantitative reverse transcription PCR (qRT-PCR) was also done to determine the mRNA level of β-catenin in treated SW480 cells. The mRNA level decreased with increasing dose, suggesting the gene silence induced by Salmonella-based tkRNAi has dose dependence (FIG. 4B).

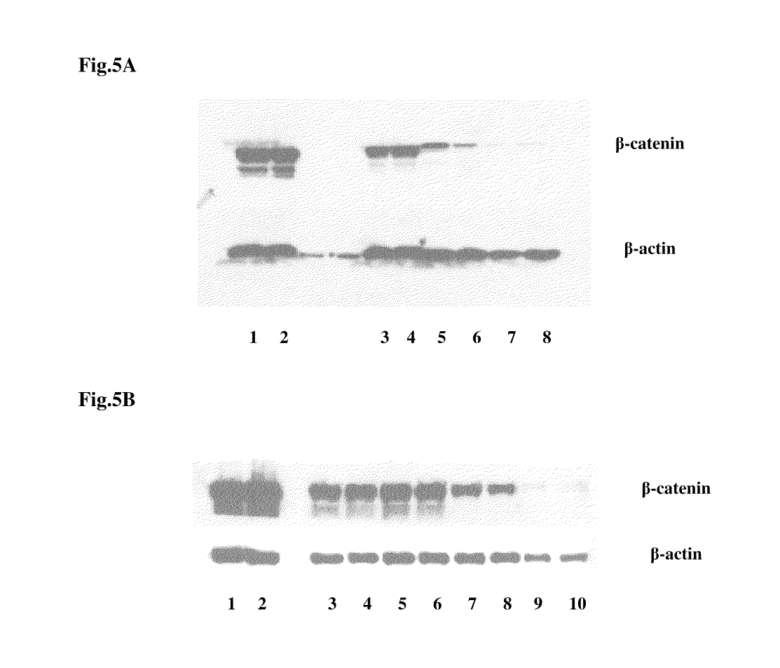

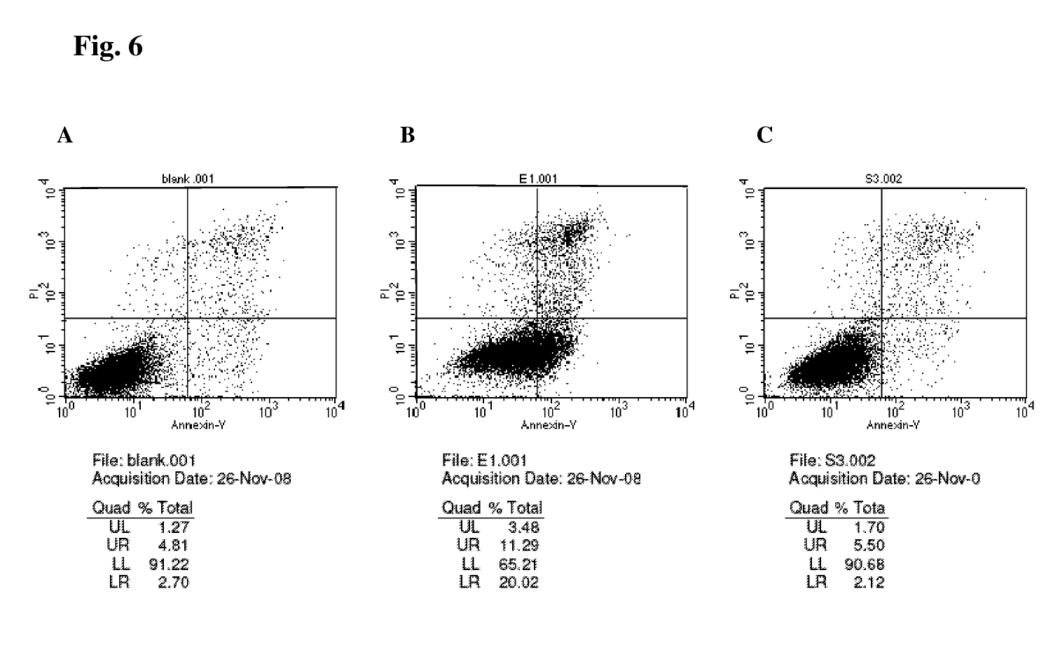

Salmonella infects cells by its own mechanism. HlyA gene was inserted into TRIPIII to determine if it could facilitate Salmonella expression of shRNA and more efficiently silence β-catenin. TRIPIIIΔHlyA was constructed similar to TRIPIII but without the HlyA gene. Also standard siRNA transfection was done to induce high silencing efficiency. After treatment, total protein was purified from SW480 and Western Blot analysis was performed. FIG. 5A shows that compared to standard siRNA transfection, SL-TRIPIIIΔHlyA-CAT induces more than 70% silencing of β-catenin and SL-TRIPIII-CAT induces around 100% silencing, suggesting that the HlyA gene increases the efficiency of gene silencing. E. Coli (BL21(DE3)) containing TRIPI-CAT (EB-TRIPI-CAT) only decreases gene expression by 50% (FIG. 5B) and induces cell death at high MOI in certain cases (FIG. 6).

Cell Apoptosis Analysis

Cell cycle analysis by flow cytometry was performed to determine cell viability after treatment with invasive bacteria. Cells were suspended and harvested 48 hr after treatment with EB-TRIPI and SL-TRIPIII. The results in FIG. 6 show that about 65% of cells survived after treatment with EB-TRIPI and that more than 90% were still alive after treatment of SL-TRIPIII.

Cell Proliferation Assay

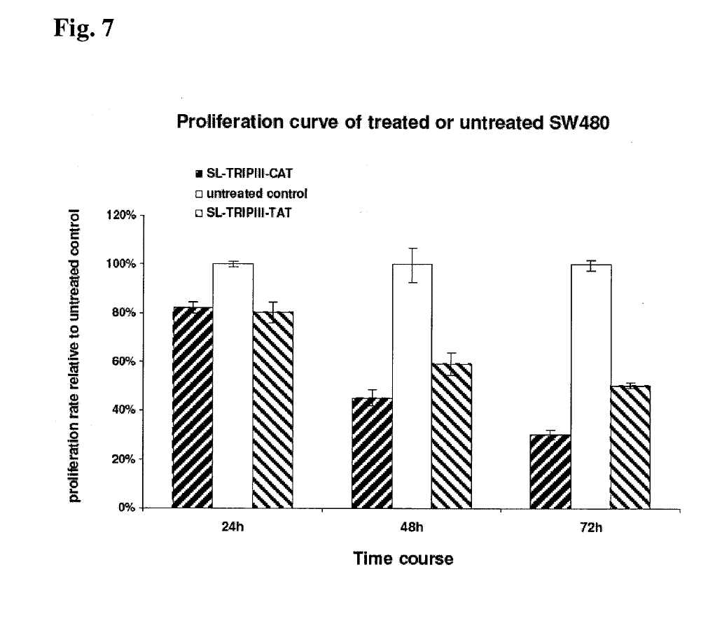

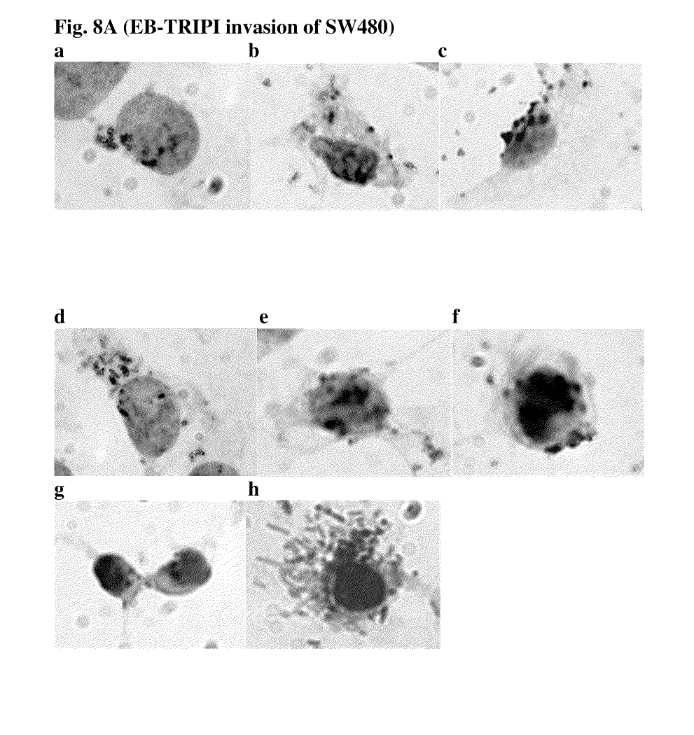

A cell proliferation assay was performed to determine whether SL-TRIPIII-CAT suppresses the growth of SW480 while silencing β-catenin. FIG. 7 shows that SL-TRIPIII-CAT treatment leads to an 80% suppression of growth of SW480 compared with cells without treatment and 40% suppression compared with treatment controls, demonstrating that SL-TRIPIII-CAT can efficiently inhibit SW480 growth by silencing the expression of β-catenin.

Bacterial Invasion

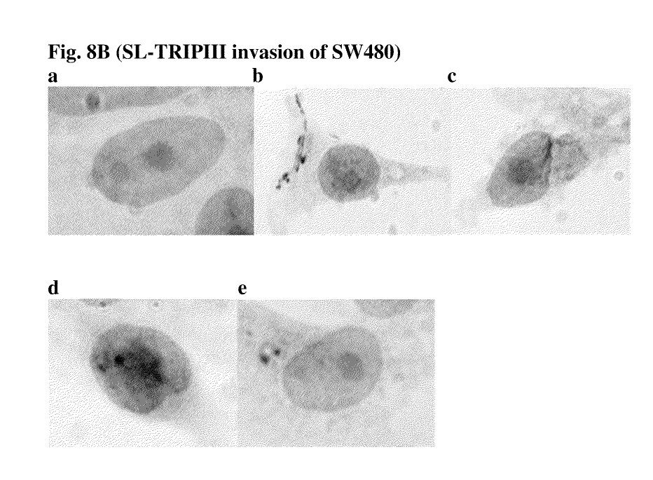

To explore how bacteria invade cells and induce cell death, SW480 cells were seeded onto Lab-Tek II chamber slides and treated with EB-TRIPI and SL-TRIPIII. The slides were fixed at different time points. Immunocyto-chemistry was performed with antibodies against HlyA or Salmonella. FIGS. 8A and 8B show Salmonella or E. coli containing TRIPIII or TRIPI invasion of target cells. In FIG. 8A, SW480 cells were treated with EB-TRIPI. FIG. 8A a-c, shows bacterial entry into the host cells. It is clear that not all E. coli are lysed and some are still intact after two days. Surviving cells not appear healthy. FIG. 8A d-g shows bacterial induction of cell death. Cell death begins with cell-shrinking and cytoplasm decreasing, continues with compression of nucleolus, and ends with cytoplasm disappearance and extreme contraction of the nucleolus. FIG. 8B a-e shows most bacteria are lysed upon invasion into cells and the lysed bacteria are clustered into a mixture. This mixture is then digested and absorbed by cells. Initially, the invaded cells display cell-shrinking and nucleolus compression. However, at the end of treatment, the cells appear to remain healthy

Treatment of Xenograft Cancer Model

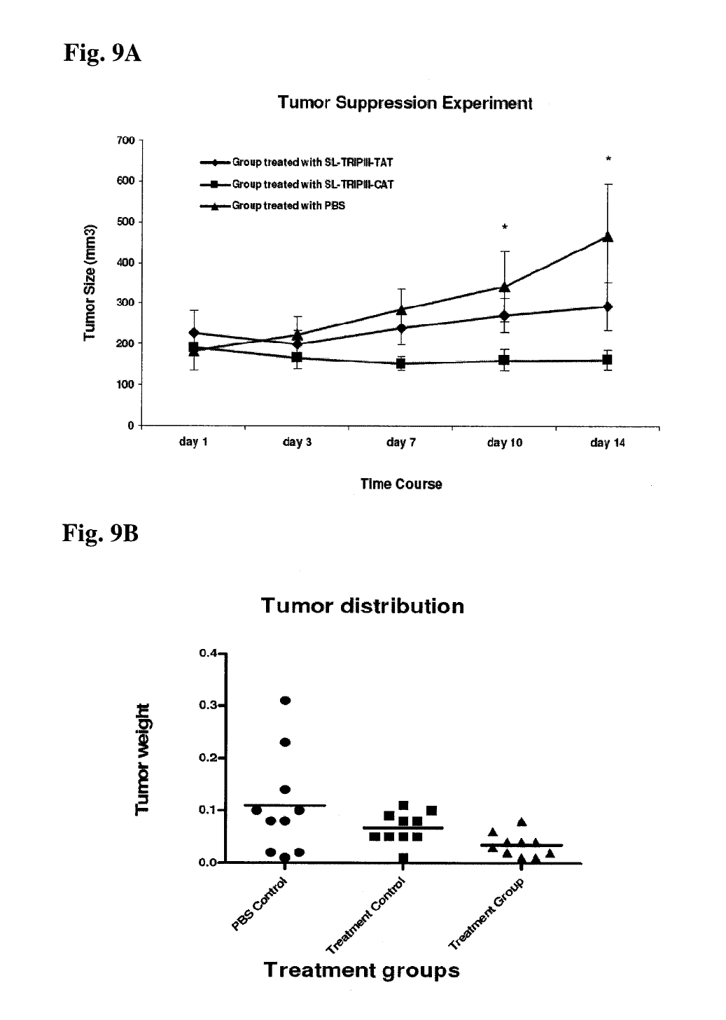

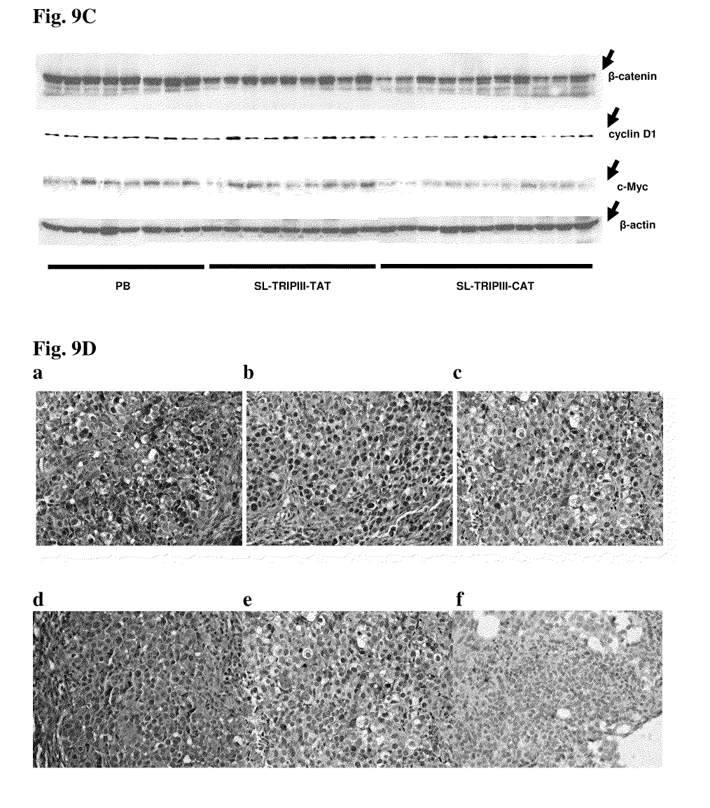

A xenograft cancer model was adopted to determine the silencing efficiency of the SL-TRIPIII system. 33 nude mice with SW480 xenograft tumors were randomized into three groups, a blank control group treated with PBS (n=10), a treatment control group treated with SL-TRIPIII-TAT (n=10), and a treatment group treated with SL-TRIPIII-CAT (n=13). Tumors treated with SL-TRIPIII-TAT were mildly suppressed compared with tumors treated with SL-TRIPIII-CAT, which showed efficient suppression of tumor development (FIG. 9A). The weight of tumors isolated from each mouse were also measured and as shown in FIG. 9B, showing the tumors' weights were greatly decreased in the treatment group compared to the blank control group and the treatment control group. Western blot analysis confirmed silencing at a molecular level showing decreased expression of β-catenin (CTNNB1) and downstream genes including c-Myc and cyclin D1 in tumors (FIG. 9C). Based on quantification of the Western blot, the β-catenin from SL-TRIPIII-CAT treatment was knocked down 70% or 50% compared with PBS treatment or SL-TRIPIII-TAT treatment respectively. There is no significant difference between PBS treatment groups and SL-TRIPIII-TAT treatment groups in the protein levels of c-Myc and cyclin D1. However the SL-TRIPIII-CAT treatment group showed 30% and 40% silencing of these two genes, respectively, indicating SL-TRIPIII-CAT can knock down not only the target gene but also genes downstream of the target gene. Immunohischemistry was also performed to check the expression of CTNNB1 in tumor sections (FIG. 9D).

Treatment of ApcMin Model

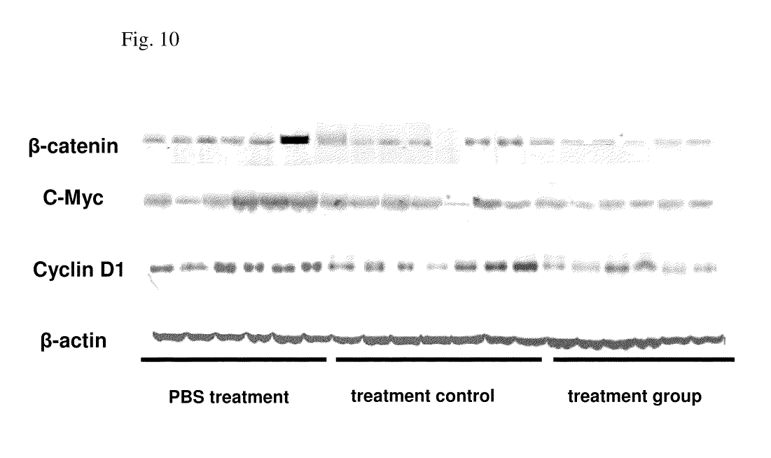

ApcMin mice were treated with SL-TRIPIII via oral administration. 19 ApcMin mice were randomized into three groups and treated with PBS (n=6), SL-TRIPIII-TAT (n=7), and SL-TRIPIII-PMC2 (n=6). Animals were treated with oral administration of 10⁸ colony forming units (cfu) of SLTRIPIII bacteria in 100 μl or equal volume of PBS every other day for 2 weeks. Animals were sacrificed and polyps were harvested from the intestines. Expression of β-catenin and its downstream genes were examined via Western blot. Western blot analysis shows very similar results to those from the xenograft cancer model (FIG. 10). The protein level of β-catenin is knocked down by 70% and 50% compared with those in PBS treatment and SL-TRIPIII-TAT treatment.

In summary, attenuated Salmonella-based tkRNAi provides a powerful tool to efficiently mediate RNAi in host cells without cellular damage. These constructs could be used in cancer therapy and make bacteria-based approaches to functional genomics possible by giving reliable phenotypes. Also, since the encoding sequence of T7 RNA polymerase was inserted into TRIPIII plasmid, this system can be applied into other naturally invasive bacteria, such as attenuated strains of Shigella and Listeria monocytogenes or even non-invasive bacteria by adding Inv gene. It therefore gives more options to explore the mechanism of bacterial delivery and to treat disease genes.

TRIPIII Plasmid Construction

HlyA expression cassette was amplified from pGB2Ωinv-hly. Oligonucleotides containing multiple cloning sites (MCS), T7 promoter, enhancer and terminator were ligated into SacI sites of KSII(+), and an shRNA encoding sequence (hairpin oligos against the CTNNB1 from human or mouse) were inserted into the BamHI and SalI sites of MCS to generate Transkindom RNA interference Plasmid III (TRIPIII).

Bacterial Culture

Plasmids were transformed into auxotrophic Salmonella typhimurium aroA 7207 (S. typhimurium 2337-65 derivative hisG46, DEL407[aroA544::Tn10(Tc-s)) (SL) by electroporation with 0.1-1 μg DNA in a 0.2 μm, 25 μF capacity cuvette, with 2 kV tension with Biorad Gene pulser indicating the time between 10 and 60 msec. Bacteria were grown at 37° C. in Brain-Heart-Infusion-broth (BHI, Remel) with addition of 100 μg/ml ampicillin (Amp). Bacterial numbers were calculated using an OD₆₀₀ measurement. For cell infection, overnight cultures were inoculated into fresh medium for another 2 hr growth. The attenuated Salmonella containing TRIPIII inserted with hairpin oligos against human or mouse CTNNB1 was named of SL-TRIPIII-CAT or SL-TRIPIII-PMC2.

Cell Culture

SW480 cells (human colon cancer cells) were cultured in RPMI1640 medium with 10% FBS supplemented with antibiotics: 100 U/ml penicillin G, 10 μg/ml streptomycin, and 2.5 μg/ml amphotericin (Sigma). For bacterial invasion, cells were cultured in 6 cm dishes to 50% confluency one day before treatment. Medium was replaced with fresh medium without serum and antibiotics 30 min before addition of bacteria. Bacteria in early log phase were washed, diluted in RPMI, and added at the desired multiplicity of infection (MOI). After exposure, cells were washed three times and fresh complete medium containing 10 μg/ml of oflaxin was added. For staining of intracellular bacteria, cells were grown on Lab-TeK II Chamber Slides (Nalgene). A 2 hr bacterial invasion was followed by a 30 min oflaxin treatment.

Western Blot

Cells were scraped off, washed, and lysed (50 mM pH 7.5 HEPES, 150 mM NaCl, 1 mM EDTA, 2.5 mM EGTA, 1% NP-40, 1 mM DTT, 1 mM PMSF and 1% Sigma protease inhibitor cocktail). 40 μg of protein was applied to 10% SDS-PAGE and transferred to 0.2 μm nitrocellulose membrane (Bio-rad). Antibodies (Santa Cruz) against CTNNB1 (sc-1496, 1:1000), β-actin (sc-47778, 1:1000), c-Myc (sc-40, 1:1000), and Cyclin D1 (sc-20044, 1:1000) were used. Protein bands were detected using ECL (Amersham).

Apoptosis Assay

Cell response after treatment was evaluated using Vybrant@ Apoptosis Assay kit#2 (Invitrogen). SW480 cells were treated for 2 hr with SL-TRIPIII and EB-TRIPI at the MOI 1:4000. Untreated cells were used as a control. The treated cells were harvested at 48 hr and were washed with PBS, and resuspended in 1× binding buffer. Cell density was determined by dilution in 1× binding buffer to 10⁶ cells/ml and preparation in 100 μl volume per assay. 5 μl of Alexa Flour@488 annexin (Competent A) and 1 μl of the 100 ug/ml PI working solution was added to each 100 μl of cell suspension. The cells were incubated at room temperature for 15 min in the dark. Finally, 400 μl of 1× binding buffer was added, and samples were evaluated using a Beckman Coulter Epics Elite ESP flow cytometer.

Cell Proliferation Assay

The proliferation inhibition of bacterial treatment to SW480 was assayed using an MTT [3-(4,5-dimethylthiazol-2-yl)-2,5-diphenyltetrazolium bromide] (Sigma) assay. 2×10⁵ cells were seeded into 96-well plates in 100 μl and grown in RPMI1640 medium (Sigma) supplemented with 10% fetal bovine serum (Sigma) at 37° C. Cells were exposed to three kinds of bacteria (SL, SL-TRIPIII-TAT, SL-TRIPIII-CAT) at the MOI 1:4000. Cells were harvested after 24 hr, 48 hr, and 72 hr of exposure and washed once with RPMI1640 medium. Cells were then incubated for 4 hr in RPMI1640 containing 1 mM MTT. The medium was then discarded, and 100 μl of dimethylsulfoxide (Sigma) was added to each well. The absorbance of formazan product was determined by ELIASA at 570 nm (Versa Max™ tunable microplate reader).

QRT-PCR

Total RNA was prepared with TRIzol regent (Invitrogen). mRNA level was determined with one-step quantitative Real-Time PCR (qRT-PCR) with a reaction kit from Applied Biosystem (Taqman Gene Expression Assay). qRT-PCR was performed on the ABI Prism 7700 Sequence Detector. Primers and probes for β-catenin and GAPDH were both from ABI. PCR reactions contained one-step Taqman Master Mix 12.5 μl, 1 μl of forward and reverse primer (100 μM), 0.5 μl of β-catenin FAM probe, and GAPDH VIC probe, 1 μl of cDNA template, and the final volume of 25 μl Cycling conditions were 50° C. for 2 min, 95° C. for 10 min, followed by 40 cycles of 95° C. for 30 sec and 60° C. for 60 sec. Relative gene expression was quantified using the comparative threshold cycle method and GAPDH as an internal standard.

Xenograft Cancer Model

33 nude mice (Charles River Laboratories) with SW480 xenograft tumors were randomized into three groups: PBS (n=10), SL-TRIPIII-TAT (n=10), and SL-TRIPIII-CAT (n=13). Animals were treated with intravenous injections of 10⁶ colony forming units (cfu) of SLTRIPIII bacteria in 100 μl or equal volume of PBS every other day for two weeks. During the in-life phase of the study, tumor sizes were measured using electronic caliper. Also, tumor weights were recorded after animals had been sacrificed. Tissues were frozen and fixed for analysis of CTNNB1 protein level by Western blotting and immunohistochemistry.

ApcMin Model

18 ApcMin mice were randomized into three groups: PBS (n=7), SL-TRIPIII-TAT (n=5), SL-TRIPIII-PMC2 (n=6). Animals were treated by oral administration of 10⁸ colony forming units (cfu) of SLTRIPIII bacteria in 100 μl or equal volume of PBS every other day for two weeks. Tissues were frozen and fixed for analysis of CTNNB1 protein level by Western Blot.

Immunohistochemistry.

Immunostaining was performed on 6 μm tissue sections using Vectastain Elite ABC avidin-biotin staining kit (Vector). For antigen retrieval, slides were heated by microwave in 5% urea. Nonspecific binding sites were blocked with 0.5% BSA for 10 min and endogenous peroxidase activity was suppressed by treatment with 3% H₂O₂ in methanol for 10 min. Sections were exposed to primary antibody CTNNB1 (sc-1496, 1:200) overnight at 4° C. The chromogen was 3,3′-diamino-enzidine (Vector); counterstaining was done with hematoxylin (Vector).

### Other Embodiments

Various modifications and variations of the described methods and compositions of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, immunology, pharmacology, endocrinology, or related fields are intended to be within the scope of the invention.

All publications mentioned in this specification are herein incorporated by reference to the same extent as if each independent publication was specifically and individually incorporated by reference.

What is claimed is:

1. A method of reducing the expression of a target gene in a cell comprising contacting said cell with live bacteria from the genus Salmonella, wherein said bacteria comprises nucleic acid sequences encoding a T7 polymerase and a T7 expression cassette, wherein said T7 expression cassette comprises a T7 promoter, a T7 terminator, and a nucleic acid sequence encoding an shRNA construct corresponding to said target gene.

2. The method of claim 1, wherein said bacteria is Salmonella typhimurium.

3. The method of claim 2, wherein said bacteria is Salmonella typhimurium aroA 7207.

4. The method of claim 1, wherein said nucleic acid construct further comprises an endosomal release factor.

5. The method of claim 4, wherein said endosomal release factor is HlyA.

6. The method of claim 1, wherein the target gene is selected from the group consisting of ABL1, β-catenin, BCL1, BCL2, BCL6, CBFA2, CBL, CSF1R, ERBA, ERBB, EBRB2, ETS1, ETS1, ETV6, FOR, FOS, FYN, HCR, HRAS, JUN, KRAS, LCK, LYN, MDM2, MLL, MYB, MYC, MYCL1, MYCN, NRAS, PIM1, PML, RET, SRC, TALI, TCL3, and YES.

7. The method of claim 1, wherein said cell is in a human subject.

8. The method of claim 7, wherein said cell is a tumor cell.

9. The method of claim 7, wherein said human has cancer.

10. The method of claim 7, wherein said cell is an intestinal cell.

11. The method of claim 7, wherein said bacteria is administered orally or intravenously.

12. The method of claim 7, wherein said human has an inflammatory disorder.

13. The method of claim 7, wherein said human has a bacterial or viral infection.

14. A nucleic acid molecule encoding a T7 polymerase, an HlyA gene, and a T7 expression cassette, wherein said T7 expression cassette comprises a T7 promoter, a T7 terminator, and a nucleic acid sequence encoding an shRNA.

15. A bacteria from the genus Salmonella, wherein said bacteria comprises the nucleic acid construct of claim 14.

16. The bacteria of claim 15, wherein said bacteria is Salmonella typhimurium

17. The bacteria of claim 16, wherein said bacteria is Salmonella typhimurium aroA 7207.

18. The bacteria of claim 15, wherein said nucleic acid construct further comprises an endosomal release factor.

19. The bacteria of claim 15, wherein said endosomal release factor is HlyA.

20. A pharmaceutical composition comprising the bacteria of claim 15.


SEQUENCE LISTING (5 sequences)

SEQ ID NO: 1 - DNA, 18 bp, Artificial Sequence
  Synthetic Construct
taatacgacacactatag

SEQ ID NO: 2 - DNA, 8 bp, Artificial Sequence
  Synthetic Construct
gagacagg

SEQ ID NO: 3 - DNA, 47 bp, Artificial Sequence
  Synthetic Construct
tagcataaccccttggggcctctaaacgggtcttgaggggttttttg

SEQ ID NO: 4 - DNA, 61 bp, Artificial Sequence
  Synthetic Construct
ggatccagctgatattgatggacagttcaagagactgtccatcaatatcagctttgtcgac

SEQ ID NO: 5 - DNA, 61 bp, Artificial Sequence
  Synthetic Construct
ggatccggaagaagatgttgacaccttcaagagaggtgtcaacatcttcttccttgtcgac